(19) United States
(12) Patent Application Publication
(10) Pub. No.: US 2010/0062066 A1
(43) Pub. Date: Mar. 11, 2010

(54) Formulations of Tetrahydropyridine Antiplatelet Agents for Parenteral or Oral Administration

(75) Inventors: Howard Bernstein, Cambridge, MA (US); Olinda Carneiro, Cambridge, MA (US); Rajeev A. Jain, Framingham, MA (US); Namrata Pandit, Watertown, MA (US); Shveta Rane, Charlotte, NC (US); Julie Ann Straub, Winchester, MA (US)

Correspondence Address: Pabst Patent Group LLP, 1545 PEACHTREE STREET NE, SUITE 320, ATLANTA, GA, 30309, US

(73) Assignee: Acusphere, Inc, Cambridge, MA (US)

(21) Appl. No.: 12/514,763

(22) PCT Filed: Nov. 8, 2007
(86) PCT No.: PCT/US07/84040

§ 371 Date: May 13, 2009

Related U.S. Application Data
(60) Provisional application No. 60/865,681, filed on Nov. 14, 2006.

(51) Int. Cl. A61K 9/113; A61K 31/4365

(52) U.S. Cl. 424/486; 424/484; 514/301

(57) ABSTRACT

A pharmaceutical composition for the oral or parenteral administration of a compound of Formula (I) comprising an oil in water emulsion, wherein the oil phase comprises the free base of or a pharmaceutically acceptable salt thereof of a compound of Formula (I), and one or more surfactants which are soluble in the oil phase and/or the aqueous phase. The emulsion optionally contains one or more excipients that are soluble in the oil phase and/or the aqueous phase, such as pH modifying agents such as buffers, osmolality/tonicity modifying agents, emulsifying agents, water-soluble polymers, and preservatives. The compound of Formula (I) can be formulated as a solid material and stored until needed. Kits for forming the emulsion are provided. Prior to administration, the solid material can be reconstituted in an aqueous medium to form the emulsion.

### CROSS-REFERENCE TO RELATED APPLICATION

This application claims priority to provisional application U.S. Ser. No. 60/865,681, entitled “Formulations of Tetrahydropyridine Antiplatelet Agents For Parenteral Or Oral Administration”, filed Nov. 14, 2006.

### FIELD OF THE INVENTION

This invention is generally in the field of tetrahydropyridine antiplatelet agent formulations for parenteral or oral administration.

### BACKGROUND OF THE INVENTION

A number of tetrahydropyridine derivatives are known to inhibit platelet aggregation. For example, U.S. Pat. Nos. 4,051,141 and 4,075,215 to Castaigne; 4,127,580 to Braye; and 4,529,596 to Aubert et al. describe tetrahydrothienopyridine derivatives; U.S. Pat. No. 4,464,377 to Blanchard et al. describes tetrahydrothienopyridine and tetrahydrofuranopyridine derivatives. Some of the more well-known derivatives include clopidogrel, prasugrel, and ticlopidine.

Clopidogrel (2-(2-chlorophenyl)-2-(2,4,5,6,7,7a-tetrahydrothieno[3,2-c]pyridine)-5-yl-acetic acetic acid methyl ester) hydrogen sulfate (Plavix®, Sanofi/BMS), is a platelet aggregation inhibitor which is described in U.S. Pat. No. 4,529,596 to Aubert et al. Clopidogrel is administered orally to patients with a history of symptomatic atherosclerotic disease, such as myocardial infarction (MI), stroke, or established peripheral vascular disease (PAD), to help prevent strokes and heart attacks by helping to prevent the formation of blood clots.

Clopidogrel is a chiral molecule and can therefore exist as the R or S enantiomer. The S-enantiomer is biologically active, while the R-enantiomer does not exhibit any anti-aggregation activity. The structure of the S-enantiomer of clopidogrel is shown below in structure 1:

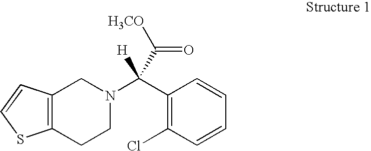

Plavix® is administered orally in tablet form, typically once a day. Oral administration, however, can be problematic for patients who are unconscious or have difficulty swallowing. Oral administration also results in delayed onset of activity since the drug has to pass through the gastrointestinal tract before being absorbed.

Prasugrel, or 2-acetoxy-5-(α-cyclopropylcarbonyl-2-fluorobenzyl)-4,5,6,7-tetrahydrothieno[3,2-c]pyridine, is described in U.S. Pat. No. 5,288,726 Koike et al. and has the structure shown below in structure 2:

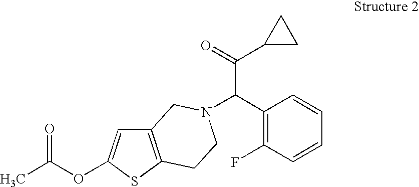

Prasugrel is an ADP receptor antagonist, which is targeted for the secondary prevention of atherothrombotic cardiovascular events in patients with acute coronary syndromes, such as unstable angina and myocardial infarction.

Ticlopidine, or 3-[(2-chlorophenyl)methyl]-7-thia-3-azabicyclo[4.3.0]nona-8,10-diene, is a platelet aggregation inhibitor structurally and pharmacologically similar to clopidogrel. When taken orally, ticlopidine causes a time- and dose-dependent inhibition of both platelet aggregation and release of platelet granule constituents. The structure of ticlopidine is shown below in structure 3:

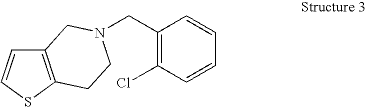

Clopidogrel formulations for parenteral administration have been reported in the literature. U.S. Pat. No. 4,847,265 to Badore et al. refers to injectable solutions of clopidogrel in an isotonic solvent. These preparations, however, typically have a pH lower than 2 which can lead to pain and irritation upon administration.

WO 2005/103059 to Sanofi-Aventis (“Sanofi”) refers to clopidogrel formulations for parenteral administration, and mentions forming aqueous, aqueous-organic, and organic solutions, suspensions, and emulsions. Examples of organic solvents or suspending media include propylene glycol, polyethylene glycol, vegetable oils, and injectable organic esters.

U.S. Patent Application Publication No. 2005/0113406 to Nagy et al. describes pharmaceutical compositions containing polymorphs of clopidogrel hydrochloride. The pharmaceutical compositions can be administered orally or parenterally. Nagy mentions a wide range of suitable oral dosage forms, including tablets, capsules, dragees (sugar coated medicated candies), solutions, elixirs, suspensions, and emulsions. The preferred liquid compositions for parenteral administration are aqueous polyethylene glycol solutions.

UK Patent Application No. GB 2 393 181 to Cipla Ltd. describes pharmaceutical compositions containing clopidogrel, or a pharmaceutically acceptable salt thereof, in amorphous form. The compositions can be administered orally or parenterally. Compositions for parenteral administration include aqueous suspensions, isotonic saline solutions, or sterile and injectable solutions.

There exists a need for improved formulations of tetrahydropyridine antiplatelet agents, which can be administered parenterally or further processed into dosage forms for other routes of administration.

Therefore, it is an object of the invention to provide improved formulations of tetrahydropyridine antiplatelet agents.

### BRIEF SUMMARY OF THE INVENTION

A pharmaceutical composition for the administration of a tetrahydropyridine anti-platelet agent in the form of an oil-in-water emulsion, methods for making the emulsion, and kits for administering the emulsion are described herein. The emulsion can be formulated for parenteral administration or further processed into an alternative dosage form such as a solid oral dosage form. The oil phase in the emulsion contains the free base of the tetrahydropyridine anti-platelet agent, or a pharmaceutically acceptable salt thereof. The emulsion also contains one or more surfactants, which are soluble in the oil phase and/or the aqueous phase of the emulsion. The emulsion optionally contains one or more excipients that are soluble in the oil phase and/or the aqueous phase, such as pH modifying agents such as buffers, osmolality/tonicity modifying agents, emulsifying agents, water-soluble polymers, and preservatives. The oil droplets in the emulsion are typically less than 10 microns in diameter, preferably less than 8 microns in diameter, more preferably less than 5 microns in diameter.

The tetrahydropyridine anti-platelet agents, and optionally, one or more excipients, can be formulated as a solid material, such as by blending and/or milling, and stored until needed. Alternatively, the anti-platelet agent, and optionally one or more excipients, can be dissolved in a solvent and then the solvent can be removed by evaporation, lyophilization, or spray drying to form a solid material. Prior to administration, the solid material may be reconstituted in an aqueous medium, optionally containing one or more excipients, to form the emulsion. After forming the emulsion, the tetrahydropyridine antiplatelet agent may be further processed to form a solid oral dosage form.

In one embodiment, the emulsion containing the tetrahydropyridine anti-platelet agent, and optionally one or more excipients, is dried and the resulting solid is formulated into a solid oral dosage form. In another embodiment, the emulsion containing the tetrahydropyridine anti-platelet agent, and optionally one or more excipients, is spray coated onto cores such as non-pareils, and the resulting solid is formulated into a solid oral dosage form. In a further embodiment, the emulsion containing the tetrahydropyridine anti-platelet agent, and optionally one or more excipients, is blended with solid excipients, extruded, and spheronized, and the resulting solid is formulated into a solid oral dosage form.

### DETAILED DESCRIPTION OF THE INVENTION

### I. Definitions

“Tetrahydropyridine anti-platelet agents” as used herein, refers to a compound containing a tetrahydropyridine ring with a thiophene, furan, or pyrrole ring fused to the tetrahydropyridine ring at the 3 and 4 positions on the pyridine ring and a phenylmethyl group on the nitrogen in the pyridine ring, derivatives, analogues and/or pharmaceutically acceptable salts thereof as shown in Formula I, provided below. “Tetrahydropyridine” and “drug” are used interchangeably herein.

“Emulsion”, as generally used herein means, a dispersion of oil and water, wherein in the oil phase exists as droplets, stabilized by the interfacial film of one or more surface active agents.

“Reconstitution medium”, “aqueous phase”, “aqueous medium”, and “water phase” are used interchangeably herein and refer to the aqueous phase of the emulsion. The oil phase is dispersed as droplets in the aqueous phase.

“Globule” or “droplet”, as generally used herein, means a small, generally spherical body of liquid.

“Pharmaceutically acceptable”, as generally used herein, refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

“Pharmaceutically acceptable salts”, as generally used herein, means derivatives of a parent compound wherein the parent compound is modified by making the acid- or base-addition salt thereof.

“Bolus”, as generally used herein, means a single dose of drug, usually injected into a blood vessel, administered over a short period of time not to exceed 60 minutes, more preferably not to exceed 30 minutes, and most preferably not to exceed 20 minutes.

“Alkanoyl”, as used herein, refers to a substituent having the following chemical formula:

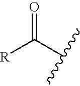

wherein R is a linear, branched, or cyclic alkyl group.

“Alkanoyloxy”, as used herein, refers to a substituent having the following chemical formula:

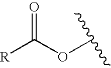

wherein R is a linear, branched, or cyclic alkyl group.

“Alkoxy carbonyl”, as used herein, refers to a substituent having the following chemical formula:

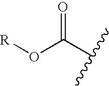

wherein R is a linear, branched, or cyclic alkyl group.

### II. Emulsion

The pharmaceutical compositions contain an oil-in-water emulsion, in a form suitable for oral or parenteral administration. Oil-in-water emulsions are dispersions of oil and water wherein in the oil phase exists as droplets, preferably microdroplets (droplets in the “micron” size range) or nanodroplets (droplets in the “nanometer” size range), stabilized by the interfacial film of one or more surface active agent(s). The droplets are small in size, with a plurality of the droplets preferably less than 10 microns in size, more preferably less than 8 microns in size, and most preferably less than 5 microns in size. In one preferred embodiment, a plurality of the droplets are less than 2 microns in size, and preferably are less than 1 micron size.

A. Oil Phase

The oil phase of the emulsion comprises the free base or a pharmaceutically acceptable salt thereof of a tetrahydropyridine anti-platelet agent of the formula shown below.

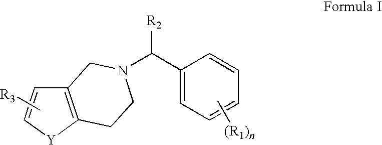

wherein

R₁ represents a hydrogen atom; an alkyl group having from 1 to 10 carbon atoms; a halogen atom; a haloalkyl group having from 1 to 10 carbon atoms and at least one halogen atom; a hydroxy group; an alkoxy group having from 1 to 10 carbon atoms; a haloalkoxy group having from 1 to 10 carbon atoms and at least one halogen atom; an alkylthio group having from 1 to 10 carbon atoms; a haloalkylthio group having from 1 to 10 carbon atoms and at least one halogen atom; an amino group; an alkanoyl group having from 1 to 10 carbon atoms; a substituted alkanoyl group which has from 2 to 10 carbon atoms and which is substituted by at least one substituent selected from the group consisting of halogen atoms, hydroxy groups, alkoxy groups having from 1 to 10 carbon atoms, and cyano groups; an alkanoyloxy group having from 1 to 10 carbon atoms; a substituted alkanoyloxy group which has from 2 to 10 carbon atoms and which is substituted by at least one substituent selected from the group consisting of halogen atoms, hydroxy groups, alkoxy groups having from 1 to 10 carbon atoms, and cyano groups; a haloalkanoyl group having from 2 to 10 carbon atoms and at least one halogen atom; a carboxy group; an alkoxycarbonyl group having from 2 to 10 carbon atoms; a substituted alkoxycarbonyl group which has from 2 to 10 carbon atoms and which is substituted by at least one substituent selected from the group consisting of halogen atoms, hydroxy groups, alkoxy groups having from 1 to 10 carbon atoms, and cyano groups; a carbamoyl group having 1 to 10 carbon atoms; a cyano group; a nitro group; an alkane-sulfonyl group having from 1 to 10 carbon atoms; a haloalkanesulfonyl group having from 1 to 10 carbon atoms and at least one halogen atom, or a sulfamoyl group;

R₂ represents hydrogen; an alkanoyl group having from 1 to 10 carbon atoms; a substituted alkanoyl group which has from 2 to 10 carbon atoms and which is substituted by at least one substituent selected from the group consisting of halogen atoms, hydroxy groups, alkoxy groups having from 1 to 10 carbon atoms, and cyano groups; an alkenoyl group having from 3 to 10 carbon atoms; a substituted alkenoyl group which has from 3 to 10 carbon atoms and which is substituted by at least one substituent selected from the group consisting of halogen atoms, hydroxy groups, alkoxy groups having from 1 to 10 carbon atoms, and cyano groups; an alkanoyloxy group having from 1 to 10 carbon atoms; a substituted alkanoyloxy group which has from 2 to 10 carbon atoms and which is substituted by at least one substituent selected from the group consisting of halogen atoms, hydroxy groups, alkoxy groups having from 1 to 10 carbon atoms, and cyano groups; an alkoxy carbonyl group having from 1 to 10 carbon atoms; a substituted alkoxycarbonyl group which is substituted by at least one substituent selected from the group consisting of halogen atoms, hydroxy groups, alkoxy groups having from 1 to 10 carbon atoms, and cyano groups; a substituted benzoyl group having at least one substituent selected from the group consisting of alkyl groups having from 1 to 10 carbon atoms, halogen atoms, and alkoxy groups having 1 to 10 carbon atoms;

R₃ represents a hydrogen atom; a hydroxy group; an alkoxy group having from 1 to 10 carbon atoms; a substituted alkoxy group which has from 1 to 10 carbon atoms and which is substituted by at least one substituent selected from the group consisting of alkoxy groups having 1 to 10 carbon atoms, alkanoyloxy groups having from 1 to 10 carbon atoms, and arylcarbonyloxy groups in which the aryl moiety has from 6 to 10 carbon atoms in a carbocyclic ring, which is unsubstituted or substituted as defined in R₁; an aralkyloxy group in which the aralkyl moiety is an alkyl group having from 1 to 10 carbon atoms which is substituted by at least one aryl group having 6 to 10 carbon atoms in a carbocyclic ring; an alkanoyloxy group having from 1 to 18 carbon atoms; an alkenyloxy group having from 3 to 10 carbon atoms; an arylcarbonyloxy group in which the aryl part has from 6 to 10 carbon atoms in a carbocyclic ring, which is unsubstituted or substituted as defined in R₁; an alkoxycarbonyloxy group having from 2 to 10 carbon atoms; an aralkyloxycarbonyloxy group in which the aralkyl part is an alkyl group having from 1 to 10 carbon atoms and which is substituted by at least one aryl group having from 6 to 10 carbon atoms in a carbocyclic ring, which is unsubstituted or substituted as defined in R₁; or a group having the formula NRₐR_{b},

in which Rₐ and R_{b} are independently selected from the group consisting of hydrogen atoms; alkyl groups having from 1 to 10 carbon atoms; substituted alkyl groups which have from 1 to 10 carbon atoms and which are substituted by at least one substituent selected from the group consisting of alkoxy groups having 1 to 10 carbon atoms, alkanoyloxy groups having from 1 to 10 carbon atoms, and arylcarbonyloxy groups in which the aryl moiety has from 6 to 10 carbon atoms in a carbocyclic ring, which is unsubstituted or substituted as defined in R₁; an aralkylamino group in which the aralkyl part is as define in R₂; an alkanoylamino group having from 1 to 18 carbon atoms; an alkenoylamino group having from 3 to 10 carbon atoms; a cycloalkyl-carbonylamino group having from 4 to 10 carbon atoms; an arylcarbonylamino group in which the aryl is an aryl group having from 6 to 10 carbon atoms in a carbocyclic ring, which is unsubstituted or substituted as defined in R₁; an alkoxycarbonylamino group having from 2 to 10 carbon atoms; an aralkyloxycarbonylamino group in which the aralkyl part is as defined in R₂;

Y represents an —NH— group, or an oxygen or sulfur atom; and

n is an integer from 1 to 5, and, when n is an integer from 2 to 5, the group represented by R₁ may be the same or different from each other.

Preferred tetrahydrothienopyridine anti-platelet agents include clopidogrel, prasugrel, and ticlopidine.

Examples of pharmaceutically acceptable salts include, but are not limited to, mineral or organic acid salts of basic residues such as amines; and alkali or organic salts of acidic residues such as carboxylic acids. The pharmaceutically acceptable salts include conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, tosylate, mesylates, besylate, and isethionic.

The pharmaceutically acceptable salts of the tetrahydropyridine anti-platelet agent can be synthesized from the parent compound, which contains a reactive moiety, by conventional chemical methods. Generally, such salts can be prepared by reacting the free base form of the tetrahydropyridine anti-platelet agent with a stoichiometric amount of the appropriate acid in water or in an organic solvent, or in a mixture of the two; generally non-aqueous media, such as ether, ethyl acetate, ethanol, isopropanol, or acetonitrile, are preferred solvents. Lists of suitable salts are found, for example, in Remington's Pharmaceutical Sciences, 20th ed., Lippincott Williams & Wilkins, Baltimore, Md., p. 704 (2000).

In one embodiment, the oil phase contains the free base of clopidogrel, or a pharmaceutically acceptable salt thereof. In another embodiment, the oil phase contains prasugrel, or a pharmaceutically acceptable salt thereof. In still another embodiment, the oil phase contains ticlopidine, or a pharmaceutically acceptable salt thereof.

B. Surfactants

The emulsion contains one or more surfactants. The surfactant can be soluble in the aqueous phase and/or the oil phase. Surfactants can be classified as anionic, cationic, amphoteric, and nonionic surfactants and include phospholipids.

Examples of suitable anionic surfactants include, but are not limited to, sodium, potassium, and ammonium salts of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate, and sodium deoxycholate.

Examples of suitable cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine.

Examples of suitable nonionic surfactants include, but are not limited to, ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates (TWEENS®), polyoxyethylene octylphenylether, PEG-1000 cetyl ether, polyoxyethylene tridecyl ether, polypropylene glycol butyl ether, POLOXAMER® 401, stearoyl monoisopropanolamide, and polyoxyethylene hydrogenated tallow amide.

Examples of amphoteric surfactants include, but are not limited to, sodium N-dodecyl-β-alanine, sodium N-lauryl-β-iminodipropionate, myristoamphoacetate, lauryl betaine and lauryl sulfobetaine.

Suitable phospholipids include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-y-alkyl phospholipids. Examples of phosphatidylcholines include such as dioleoylphosphatidylcholine, dimyristoylphosphatidylcholine (DMPC), dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcholine (DBPC), ditricosanoyl-phosphatidylcholine (DTPC), dilignoceroylphatidylcholine (DLPC); and phosphatidylethanolamines such as dioleoylphosphatidylethanolamine or 1-hexadecyl-2-palmitoyl glycerophospho-ethanolamine. Synthetic phospholipids with asymmetric acyl chains (e.g., with one acyl chain of 6 carbons and another acyl chain of 12 carbons) may also be used.

Examples of phosphatidylethanol-amines include, but are not limited to, dicaprylphosphatidylethanolamine, dioctanoylphosphatidyl-ethanolamine, dilauroylphosphatidylethanolamine, dimyristoylphosphatidyl-ethanolamine (DMPE), dipalmitoylphosphatidylethanolamine (DPPE), dipalmitoleoylphosphatidylethanolamine, distearoylphosphatidylethanolamine (DSPE), dioleoylphosphatidylethanolamine, and dilineoylphosphatidylethanol-amine.

Examples of phosphatidylglycerols include, but are not limited to, dicaprylphosphatidylglycerol, dioctanoylphosphatidylglycerol, dilauroylphosphatidylglycerol, dimyristoylphosphatidylglycerol (DMPG), dipalmitoylphosphatidylglycerol (DPPG), dipalmitoleoylphosphatidylglycerol, distearoylphosphatidylglycerol (DSPG), dioleoylphosphatidylglyeerol, and dilineoylphosphatidylglycerol.

In a preferred embodiment, the surfactant is a polysorbate.

C. Aqueous Phase

The aqueous phase, also referred to herein as the “reconstitution medium”, “aqueous medium”, or “water phase” is typically water or an aqueous buffer. Optionally, the aqueous phase also contains one or more co-solvents which are miscible with water, such as lower alcohols (e.g. ethanol, propylene glycol, etc).

D. Other Excipients

The emulsion optionally further contains one or more excipients that are soluble in the oil phase and/or aqueous phase of the emulsion, including, but not limited to, salts, buffers, pH modifying agents, emulsifiers, preservatives, anti-oxidants, osmolality/tonicity modifying agents, and water-soluble polymers.

The emulsion is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, and thimerosal.

In one embodiment, the emulsion contains polyvinylpyrrolidone.

### II. Method of Making the Emulsion

A. Tetrahydropyridine Antiplatelet Agent Added as an Oil

An emulsion can be formed by adding the tetrahydropyridine anti-platelet agent, as an oil, to the aqueous phase to form the emulsion. Optionally, the mixture is shaken or agitated to form the emulsion. The surfactant and, optionally, other excipients can be dissolved in the oil phase and/or the aqueous phase. Suitable excipients include, but are not limited to, salts, sugars, buffers, pH modifying agents, emulsifiers, preservatives, anti-oxidants, osmolality/tonicity modifying agents, water-soluble polymers, and combinations thereof. The aqueous phase typically contains water but can contain one or more co-solvents which are miscible with water, such as lower alcohols (e.g. ethanol, propylene glycol, etc.). The concentration of the anti-platelet agent is from about 1 to about 100 mg/ml of reconstitution medium, preferably from about 10 to about 100 mg/ml of reconstitution medium, and more preferably from about 30 to about 60 mg/ml of reconstitution medium.

B. Tetrahydropyridine Antiplatelet Added as a Solid

An emulsion can be formed by adding the tetrahydropyridine anti-platelet agent, or a pharmaceutically acceptable salt thereof, as a solid, to the reconstitution medium and shaking or agitating the suspension until the emulsion forms. The aqueous phase typically contains water but can contain one or more co-solvents which are miscible with water, such as lower alcohols (e.g. ethanol, propylene glycol, etc.). The concentration of the anti-platelet agent is from about 1 to about 100 mg/ml of reconstitution medium, preferably from about 10 to about 100 mg/ml of reconstitution medium, and more preferably from about 30 to about 60 mg/ml of reconstitution medium.

The solid tetrahydropyridine antiplatelet agent can be the raw, dry crystals obtained from a supplier without modification. Alternatively, the solid tetrahydropyridine antiplatelet agent can be blended with one or more excipients. The blend can optionally be milled using techniques known in the art, such as jet milling.

Alternatively, the raw tetrahydropyridine antiplatelet agent can be dissolved in a solvent, optionally with one or more excipients, followed by removal of the solvent, to prepare a solid. Techniques for removing the solvent are well-known in the art and include evaporation, lyophilization, and spray drying. Suitable excipients include, but are not limited to, salts, sugars, buffers, pH modifying agents, emulsifiers, preservatives, anti-oxidants, osmolality/tonicity modifying agents, water-soluble polymers, and combinations thereof. Following solvent removal, the solid material can be milled by techniques known in the art, such as jet milling.

In one embodiment, mannitol is jet milled with clopidogrel bisulfate, and the resulting solid material is reconstituted in an aqueous medium containing acetate buffer, a surfactant (such as TWEEN® 80), and a water-soluble polymer (such as Plasdone C-15) to form an emulsion.

In another embodiment, a solution of clopidogrel bisulfate, polysorbate 80, and optionally mannitol, is lyophilized to form a solid matrix. The matrix is then reconstituted in an aqueous medium containing buffer, to form the emulsion, such that the pH of the emulsion is suitable for parenteral administration (pH in the range of 3 to 8). The initial solution of clopidogrel bisulfate, polysorbate 80, and optionally mannitol is not suitable for parenteral administration due to the low pH of clopidogrel bisulfate.

C. Formation of Solid Oral Dosage Forms

In another embodiment, the anti-platelet agent, a surfactant, and optionally one or more excipients are processed into an emulsion. Then the emulsion may be processed using methods such as lyophilization, spray drying or spray coating onto cores such as non-pareils to form a solid. The solid can then be formulated into a solid oral dosage form, such as, a powder or pellet filled capsule, a wafer or a film, a modified or targeted delivery tablet or an orally disintegrating tablet. In a preferred embodiment, the surfactant is a polysorbate, such as polysorbate 80.

In still another embodiment, the anti-platelet agent, a surfactant, and optionally one or more excipients, are processed into an emulsion. Then the emulsion may be blended with solid excipients, and subsequently processed using methods such as extrusion and spheronization, or granulation, to form a solid. The solid can be formulated into a solid oral dosage form, such as, a powder or pellet filled capsule, a wafer or a film, a modified or targeted delivery tablet or an orally disintegrating tablet. In a preferred embodiment, the surfactant is a polysorbate, such as polysorbate 80.

Tablets are solid pharmaceutical dosage forms containing a pharmaceutical agent, with or without suitable excipients and are prepared by compression or molding methods. The antiplatelet agents can be processed into tablets using standard tabletting methods. Compressed tablets are prepared using a tablet press from powders or granules in combination with excipients such as diluents, binders, disintegrants, lubricants, and glidants. Other excipients like modified release polymers, waxes, colors, sweeteners or flavors can also be added. Tablets or capsules can be further coated with polymer or sugar films or enteric or sustained release polymer coatings. Layered tablets can be prepared by compressing additional powders or granules on a previously prepared tablet for immediate or modified release. Powders can be processed into granules using wet granulation methods, dry granulation methods, melt extrusion or spray drying of the powder dispersed into an appropriate liquid. The granules can be filled into capsules, processed into tablets or further processed into pellets using spheronization equipment. Pellets can be directly filled into capsules or compressed into tablets.

In still another embodiment, the anti-platelet agent, a surfactant, and optionally excipients are processed into a solid (e.g., through blending and milling, or through solubilization and drying), which may be formulated into an enteric coated solid oral dosage form. Following oral administration to a patient, this solid oral dosage form may produce an emulsion in vivo in the intestinal tract. In a preferred embodiment, the surfactant is a polysorbate, such as polysorbate 80.

### III. Method of Administering the Emulsion

The emulsion is typically prepared immediately prior to use. However, the emulsion may be prepared up to 5 years prior to use and stored until needed. The solid starting material is suspended in a reconstitution medium, and the mixture is agitated to form the emulsion.

The emulsion may be formed using a kit. The kit generally contains (1) the tetrahydropyridine antiplatelet agent or a pharmaceutically acceptable salt thereof, (2) a surfactant that is soluble in the oil phase and/or the aqueous phase, and (3) an aqueous phase. Optionally the kit includes one or more excipients, which are stored with the tetrahydropyridine antiplatelet agent, the surfactant, and/or the aqueous phase. The tetrahydropyridine antiplatelet agent may be in a solid or liquid form. If it is in a liquid form, the tetrahydropyridine antiplatelet agent is in an oil phase. In one embodiment, the kit is in the form of a syringe, such as a mixing syringe, which contains a barrier means to prevent the admixing of the solid/oil phases and aqueous phases until use is desired. The tetrahydropyridine as a solid or in an oil phase and the aqueous phase are separated by the barrier prior to use. To form an emulsion, the user activates the mixing syringe in its intended manner to remove the barrier, to mix the tetrahydropyridine antiplatelet agent (in solid form or as an oil) with the aqueous phase and form the emulsion.

The emulsion is typically administered to a patient via intravenous administration as a bolus. The dosage is dependent on the specific drug to be administered, the patient to be treated, and the disease to be treated and can be readily determined by one of ordinary skill in the art, such as the attending physician. Typical dosages range from about 1 mg to about 2000 mg.

The present invention will be further understood by reference to the following non-limiting examples.

### EXAMPLES

### Materials

Materials were obtained from the following sources: clopidogrel bisulfate (Onbio, Richmond Hill, Ontario, Canada), mannitol (Pearlitol® 100SD from Roquette America Inc., Keokuk, Iowa), polysorbate 80 (TWEEN® 80, Spectrum Chemicals, New Brunswick, N.J.), Plasdone C15 (International Specialty Products Technologies, Wayne, N.J.), sodium acetate (Spectrum Chemicals, New Brunswick, N.J.), and sodium hydroxide (Sigma Chemical Co., St. Louis, Mo.). TWEEN® 80 is hereinafter referred to as “Tween 80.”

A TURBULA® inversion mixer (model: T2F) was used for blending. A “Fluid Energy Jet-O-Mizer™ Model 00” jet mill was used for milling. Dry nitrogen gas was used as the injector and grinding gases during milling. In the examples, the dry powder was fed manually into the jet mill. Thus, the powder feed rate was not constant. Although the powder feeding was manual, the feed rate was calculated to be approximately in the range of 1.0-3.0 g/min. for all of the examples. Feed rate is the ratio of the mass of the total material processed in one batch to the total batch time.

### Example 1

### Jet Milling Clopidogrel Bisulfate

The clopidogrel bisulfate powder was fed manually into the Fluid Energy jet mill, with an injector pressure of 8 bars and a grinding pressure of 4 bars. The jet mill was allowed to clear out for 1 minute with an injector pressure of 10 bars and a grinding pressure of 9 bar resulting in jet milled clopidogrel bisulfate.

### Example 2

### Jet Milling of a Blend of Clopidogrel Bisulfate and Mannitol

Clopidogrel bisulfate (3.3502 g) and mannitol (6.678 g) were blended together in the TURBULA® mixer at 96 rpm for 10 min. The resultant powder blend was jet milled in the Fluid Energy jet mill with an injector pressure of 8 bars and a grinding pressure of 4 bars. The jet mill was allowed to clear out for 1 minute with an injector pressure of 10 bars and a grinding pressure of 9 bars.

### Example 3

### Comparison of Reconstituted Jet Milled Clopidogrel Bisulfate

The jet milled clopidogrel bisulfate produced in Example 1 was mixed using shaking at a concentration of 10 mg clopidogrel bisulfate/mL reconstitution medium using each of the following four (4) media:

(1) 0.1 M sodium acetate,

(2) 0.1 M sodium acetate with Plasdone C15 at 5 mg/mL and Tween 80 at 5 mg/mL,

(3) 0.1 M sodium acetate with Tween 80 at 5 mg/mL, and

(4) 0.1 M sodium acetate with Plasdone C15 at 5 mg/mL.

The resulting materials were analyzed by visual evaluation and light microscopy.

Visual observations showed that fine emulsions were formed when the reconstitution medium contained Tween 80 (Samples 2 and 3). Light microscopy images were taken of the materials described above. The images showed that the oil phase, which contained clopidogrel, existed as round droplets or globules dispersed within the aqueous medium. Emulsions did not form in Samples 1 and 4.

### Example 4

### Comparison of Reconstituted Jet Milled Blend of Clopidogrel Bisulfate and Mannitol

The jet milled blend of clopidogrel bisulfate and mannitol produced in Example 2 was mixed using shaking at a concentration of 10 mg clopidogrel bisulfate/mL reconstitution medium using each of the following four (4) media:

(1) 0.1 M sodium acetate,

(2) 0.1 M sodium acetate with Plasdone C15 at 5 mg/mL and Tween 80 at 5 mg/mL,

(3) 0.1 M sodium acetate with Tween 80 at 5 mg/mL, and

(4) 0.1 M sodium acetate with Plasdone C15 at 5 mg/mL.

The resulting materials were analyzed by visual evaluation and light microscopy.

Visual observations showed that fine emulsions formed when the reconstitution vehicle contained Tween 80 (Samples 2 and 3), Light microscopy images were taken of the materials described above. The images showed that the oil phase, which contained clopidogrel, existed as round droplets or globules dispersed within the aqueous vehicle. Emulsions did not form in Samples 1 and 4.

### Example 5

### Lyophilization of Clopidogrel Bisulfate and Polysorbate 80

Clopidogrel bisulfate (6 g) was added to 100 mL of an aqueous solution of Tween 80 (5 mg/mL in water). The mixture was sonicated for 15 minutes, and then brought to 150 mL total volume using an aqueous solution of Tween 80 (5 mg/mL in water). A sample (80 mL) was filtered through a 0.2 μm PES filter, and then dispensed in 5 mL aliquots into 20 mL vials. The vials were frozen at −80° C., and lyophilized on a Virtis lyophilizer.

The resultant vials containing lyophilized solid were reconstituted with 5 mL of an aqueous solution containing 0.1 M sodium acetate and 0.19 M sodium hydroxide, and the vials shaken. A pH 6.16 milky white emulsion was formed upon reconstitution. Light microscopy images were taken of the emulsion described above. The images showed that the oil phase, which contained clopidogrel, existed as round droplets or globules dispersed within the reconstitution medium.

Unless defined otherwise, all technical and scientific terms used herein have the same meanings as commonly understood by one of skill in the art to which the disclosed invention belongs.

Those skilled in the art will recognize, or be able to ascertain using no more than routine experimentation, many equivalents to the specific embodiments of the invention described herein. Such equivalents are intended to be encompassed by the following claims.

1. A pharmaceutical composition comprising an oil-in-water emulsion comprising an oil phase and an aqueous phase wherein the emulsion comprises a surfactant that is soluble in the oil phase and/or the aqueous phase,
wherein the oil phase comprises the free base or a pharmaceutically acceptable salt thereof of a compound of Formula I, 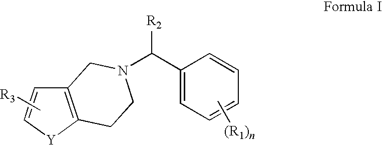
wherein
R₁ represents a hydrogen atom; an alkyl group having from 1 to 10 carbon atoms; a halogen atom; a haloalkyl group having from 1 to 10 carbon atoms and at least one halogen atom; a hydroxy group; an alkoxy group having from 1 to 10 carbon atoms; a haloalkoxy group having from 1 to 10 carbon atoms and at least one halogen atom; an alkylthio group having from 1 to 10 carbon atoms; a haloalkylthio group having from 1 to 10 carbon atoms and at least one halogen atom; an amino group; an alkanoyl group having from 1 to 10 carbon atoms; a substituted alkanoyl group which has from 2 to 10 carbon atoms and which is substituted by at least one substituent selected from the group consisting of halogen atoms, hydroxy groups, alkoxy groups having from 1 to 10 carbon atoms, and cyano groups; an alkanoyloxy group having from 1 to 10 carbon atoms; a substituted alkanoyloxy group which has from 2 to 10 carbon atoms and which is substituted by at least one substituent selected from the group consisting of halogen atoms, hydroxy groups, alkoxy groups having from 1 to 10 carbon atoms, and cyano groups; a haloalkanoyl group having from 2 to 10 carbon atoms and at least one halogen atom; a carboxy group; an alkoxycarbonyl group having from 2 to 10 carbon atoms; a substituted alkoxycarbonyl group which has from 2 to 10 carbon atoms and which is substituted by at least one substituent selected from the group consisting of halogen atoms, hydroxy groups, alkoxy groups having from 1 to 10 carbon atoms, and cyano groups; a carbamoyl group having 1 to 10 carbon atoms; a cyano group; a nitro group; an alkane-sulfonyl group having from 1 to 10 carbon atoms; a haloalkanesulfonyl group having from 1 to 10 carbon atoms and at least one halogen atom, or a sulfamoyl group;
R₂ represents hydrogen; an alkanoyl group having from 1 to 10 carbon atoms; a substituted alkanoyl group which has from 2 to 10 carbon atoms and which is substituted by at least one substituent selected from the group consisting of halogen atoms, hydroxy groups, alkoxy groups having from 1 to 10 carbon atoms, and cyano groups; an alkenoyl group having from 3 to 10 carbon atoms; a substituted alkenoyl group which has from 3 to 10 carbon atoms and which is substituted by at least one substituent selected from the group consisting of halogen atoms, hydroxy groups, alkoxy groups having from 1 to 10 carbon atoms, and cyano groups; an alkanoyloxy group having from 1 to 10 carbon atoms; a substituted alkanoyloxy group which has from 2 to 10 carbon atoms and which is substituted by at least one substituent selected from the group consisting of halogen atoms, hydroxy groups, alkoxy groups having from 1 to 10 carbon atoms, and cyano groups; an alkoxy carbonyl group having from 1 to 10 carbon atoms; a substituted alkoxycarbonyl group which is substituted by at least one substituent selected from the group consisting of halogen atoms, hydroxy groups, alkoxy groups having from 1 to 10 carbon atoms, and cyano groups; a substituted benzoyl group having at least one substituent selected from the group consisting of alkyl groups having from 1 to 10 carbon atoms, halogen atoms, and alkoxy groups having 1 to 10 carbon atoms;
R₃ represents a hydrogen atom; a hydroxy group; an alkoxy group having from 1 to 10 carbon atoms; a substituted alkoxy group which has from 1 to 10 carbon atoms and which is substituted by at least one substituent selected from the group consisting of alkoxy groups having 1 to 10 carbon atoms, alkanoyloxy groups having from 1 to 10 carbon atoms, and arylcarbonyloxy groups in which the aryl moiety has from 6 to 10 carbon atoms in a carbocyclic ring, which is unsubstituted or substituted as defined in R₁; an aralkyloxy group in which the aralkyl moiety is an alkyl group having from 1 to 10 carbon atoms which is substituted by at least one aryl group having 6 to 10 carbon atoms in a carbocyclic ring; an alkanoyloxy group having from 1 to 18 carbon atoms; an alkenyloxy group having from 3 to 10 carbon atoms; an arylcarbonyloxy group in which the aryl part has from 6 to 10 carbon atoms in a carbocyclic ring, which is unsubstituted or substituted as defined in R₁; an alkoxycarbonyloxy group having from 2 to 10 carbon atoms; an aralkyloxycarbonyloxy group in which the aralkyl part is an alkyl group having from 1 to 10 carbon atoms and which is substituted by at least one aryl group having from 6 to 10 carbon atoms in a carbocyclic ring, which is unsubstituted or substituted as defined in R₁; or a group having the formula —NRₐR_{b},
in which Rₐ and R_{b} are independently selected from the group consisting of hydrogen atoms; alkyl groups having from 1 to 10 carbon atoms; substituted alkyl groups which have from 1 to 10 carbon atoms and which are substituted by at least one substituent selected from the group consisting of alkoxy groups having 1 to 10 carbon atoms, alkanoyloxy groups having from 1 to 10 carbon atoms, and arylcarbonyloxy groups in which the aryl moiety has from 6 to 10 carbon atoms in a carbocyclic ring, which is unsubstituted or substituted as defined in R₁; an aralkylamino group in which the aralkyl part is as define in R₂; an alkanoylamino group having from 1 to 18 carbon atoms; an alkenoylamino group having from 3 to 10 carbon atoms; a cycloalkyl-carbonylamino group having from 4 to 10 carbon atoms; an arylcarbonylamino group in which the aryl is an aryl group having from 6 to 10 carbon atoms in a carbocyclic ring, which is unsubstituted or substituted as defined in R₁; an alkoxycarbonylamino group having from 2 to 10 carbon atoms; an aralkyloxycarbonylamino group in which the aralkyl part is as defined in R₂;
Y represents an —NH— group, or an oxygen or sulfur atom; and
n is an integer from 1 to 5, and, when n is an integer from 2 to 5, the group represented by R₁ may be the same or different from each other;
wherein the composition is in a form suitable for parenteral or oral administration.

2. The composition of claim 1, wherein the compound of formula I is clopidogrel.

3. The composition of claim 1, wherein the compound of formula I is selected from the group consisting of prasugrel and ticlopidine.

4. (canceled)

5. The composition of claim 1, wherein the concentration of the compound of formula I is from about 1 to about 100 mg/ml of the aqueous phase.

6. (canceled)

7. (canceled)

8. The composition of claim 1, wherein the surfactant is selected from the group consisting of anionic, cationic, amphoteric, and nonionic surfactants.

9. The composition of claim 1, wherein the surfactant is a polysorbate.

10. The composition of claim 1, wherein the concentration of the surfactant is from about 0.005 to about 50 mg/ml of the aqueous phase.

11. (canceled)

12. The composition of claim 1, wherein the emulsion further comprises one or more excipients selected from the group consisting of emulsifiers, water-soluble polymers, pH modifying agents, anti-oxidants, osmolality/tonicity modifying agents, preservatives, and combinations thereof.

13. The composition of claim 12, wherein the one or more excipients are soluble in the oil phase and/or the aqueous phase.

14. The composition of claim 12, wherein the one or more excipients is a water-soluble polymer.

15. The composition of claim 14, wherein the water-soluble polymer is polyvinylpyrrolidone.

16. The composition of claim 14, wherein the concentration of the polyvinylpyrrolidone is from about 0.005 to about 20 mg/ml of the aqueous phase.

17. The composition of claim 1, wherein the composition is in a form suitable for parenteral administration.

18. A method of making the composition of claim 1, comprising
suspending a solid comprising a compound of Formula I or a pharmaceutically acceptable salt thereof in a sterile aqueous medium to form a mixture,
and agitating the mixture to form an emulsion.

19. The method of claim 18, wherein the solid is formed by a technique selected from the group consisting of spray drying, lyophilizing, and precipitation.

20. The method of claim 18, wherein the solid further comprises one or more excipients.

21. The method of claim 20, wherein the one or more excipients is selected from the group consisting of salts, pH modifying agents such as buffers, emulsifiers, anti-oxidants, preservatives, osmolality/tonicity modifying agents, and surfactants.

22. The method of claim 20, wherein the excipient is an osmolality/tonicity modifying agent selected from the group consisting of salts and sugars.

23. The method of claim 22, wherein the osmolality/tonicity modifying agents is a sugar.

24. A method of treatment comprising parenterally administering to a patient in need thereof the composition of claim 1 comprising an effective amount of the compound of Formula I or pharmaceutically acceptable salt thereof.

25. The method of claim 24, wherein the composition is administered as a bolus.

26. A kit for forming the composition of claim 1, comprising
(i) the oil phase comprising the free base of the compound of Formula I or a pharmaceutically acceptable salt thereof,
(ii) the surfactant, and
(iii) the aqueous phase.

27. The kit of claim 26, further comprising a syringe, wherein the oil phase and the aqueous phase are separated by a barrier in the syringe.

28. The kit of claim 26, wherein the oil phase further comprises one or more excipients.

29. The kit of claim 26, wherein the aqueous phase further comprises one or more excipients.

30. A kit for forming the composition of claim 1, comprising
(i) a solid comprising the free base of the compound of Formula I or pharmaceutically acceptable salt thereof,
(ii) the surfactant, and
(iii) the aqueous phase.

31. The kit of claim 30, further comprising a syringe, wherein the solid and the aqueous phase are separated by a barrier in the syringe.

32. The kit of claim 30, wherein the solid material further comprises one or more excipients.

33. The kit of claim 30, wherein the aqueous phase further comprises one or more excipients.

34. (canceled)

35. (canceled)

36. (canceled)
